(19) United States
(12) Patent Application Publication
(10) Pub. No.: US 2020/0375605 A1
(43) Pub. Date: Dec. 3, 2020

(54) A COLLAPSIBLE AND ADJUSTABLE VESSEL TREATMENT DEVICE AND ADVANCED CUFF WITH INDEPENDENT AND DYNAMICALLY CONTROLLED CHARGE AND DISCHARGE MODES FOR A VESSEL OR SAC WALL TREATMENT AND A CARDIAC ASSIST DEVICE

(71) Applicant: BIOQ DEVICES PTY LTD, Eventon Park (AU)

(72) Inventors: Peter William WALSH, Everton Park (AU); Adrian Jeffery LOWRY, Nerang (AU); Madhusudanrao NEELI, Kuraby (AU); David ROMERO, Taringa (AU); Jorge Alberto AMAYA CATAÑO, New Farm (AU)

(21) Appl. No.: 16/970,656

(22) PCT Filed: Feb. 21, 2019
(86) PCT No.: PCT/AU2019/000021

§ 371 Date: Aug. 18, 2020

(30) Foreign Application Priority Data
Feb. 20, 2018 (AU) 2018900533

(51) Int. Cl. A61B 17/12; A61M 1/12; A61B 5/0215

(52) U.S. Cl. CPC A61B 17/12136; A61M 1/127; A61B 5/0215; A61M 2205/8206; A61M 2205/8243; A61M 2205/3344; A61M 2205/3317; A61M 2205/073

(57) ABSTRACT

A method of treating a vessel in a human or animal body, including the steps of: positioning an implantable device against a portion of tubular or sac wall of the vessel, whereby a load applied to the vessel is borne by the vessel wall and also by the device to transfer energy to an energy storage means, the vessel being assisted when the energy storage means returns the stored energy to the device. Further disclosed is a treatment or assistance device for operating in or with a tubular or sac wall of a vessel in a human or animal body, the device including a changeable volume portion which is adapted to interact with the vessel to modify the vessel's volume; and an energy storage means functioning with the changeable volume portion whereby a decrease in the volume of said changeable volume portion creates an energy charge in the energy storage means, the energy charge being able to be subsequently released to cause the changeable volume portion to increase in volume. Improved cuff features for stable attachment with monitoring capabilities have been described as has dynamically controlling the charge and discharge phases passively, with control electronics, and with energy harvesting.

### CROSS-REFERENCE TO RELATED APPLICATIONS

This application is a 35 U.S.C. §371 national phase application of PCT/AU2019/000021 filed Feb. 21, 2019 entitled “A COLLAPSIBLE AND ADJUSTABLE VESSEL TREATMENT DEVICE AND ADVANCED CUFF WITH INDEPENDENT AND DYNAMICALLY CONTROLLED CHARGE AND DISCHARGE MODES FOR A VESSEL OR SAC WALL TREATMENT AND A CARDIAC ASSIST DEVICE,” which claims the benefit of and priority to Australian Patent Application No. 2018900533 filed Feb.20, 2018, the contents of which being incorporated by reference in their entireties herein.

### FIELD OF THE INVENTION

The present invention relates to tubular wall compliance and load bearing devices and methods for their deployment within human and or animal bodies, so as to change or modify the compliance or the load bearing capacity of a tubular or sac wall section.

When applied to the cardiovascular system, these inventions serve to boost the secondary heart pump action of the heart, by dampening the time dependent blood pressure profile during systole, and enhancing the time dependent blood pressure profile during diastole, thereby reducing heart load and improving aortic and coronary artery blood flow.

### BACKGROUND

Heart failure is the fastest growing cardiovascular disorder. Incidence is rising at a rate of approximately 2% to 5% in people over 65 years of age, and 10% in people over 75 years of age.

Heart failure is a leading cause of hospital admissions and re-admissions in Americans older than 65 years of age.

Hypertension is a common condition prior to heart failure. In a recent study; 91% of people who developed heart failure had previous hypertension, of which 42% had systolic dysfunction and 58% had diastolic dysfunction.

Aortic stiffening, due to elastin degradation and other forms of stiffening, such as that caused by atherosclerosis, which is stiffening due to the presence and buildup of plaques, are a cause of hypertension. The aorta stiffens and dilates with age increasing: the load on the heart; pressure in left ventricle; aortic pressure at the time of peak aortic flow, and pulse wave velocity in the aorta and early wave reflection thus increasing pressure in late systole.

Data shows that systolic blood pressure continues to rise with age and diastolic pressure remains constant after approximately 50 years of age, giving an increase in pulse pressure after 50 years of age.

As the aorta stiffens, the arterial system suffers from a lack of compliance, leading to hypertension. Therefore aortic stiffening appears to be a factor leading to heart failure.

Aortic compliance is fundamental to effective cardiovascular dynamics. Lack of aortic compliance leads to increased heart loading during systole and poor coronary artery perfusion during diastole due to a lack of vessel recoil. Decreases in aortic compliance occur with age as a result of stiffening in the aortic wall. Approximately 80% of arterial compliance is in the ascending aorta and aortic arch sections. This expansion during systole and contraction/recoil during diastole of the ascending aorta and arch, is referred to as the secondary heart pump; an action that decays with age and disease.

Stiffness of the aortic wall can be defined using various measures, and is commonly expressed as the pressure-strain elastic modulus, Eₚ:

Eₚ=D_{dia}×(D_{sys}−D_{dia})/(P_{sys}−P_{dia})

Where D_{sys} and D_{dia} and the diameter of the vessel in systole and diastole respectively, and P_{sys} and P_{dia} are the pressure within the vessel at systole and diastole respectively.

Aortic stiffening is generally associated with vessel dilation. Previous solutions for addressing heart failure include: (a) medications which have limited benefits and generally high costs associated with them; (b) intra-aortic balloons which are only a temporary solution; (c) ventricular assist devices, extraluminal and intraluminal compression devices, and pumps, which require power sources thereby increasing complexity of implanting, increase expense and have higher risk to the patient; and (d) heart transplants which are limited by availability, high cost, and high risk.

The applicant does not concede that the prior art discussed in the specification forms part of the common general knowledge in the art at the priority date of this application.

### BRIEF SUMMARY OF THE INVENTION

The present invention provides a method of treating a vessel in a human or animal body, said method including the steps of: preparing a patient; identifying a site in said vessel requiring treatment; positioning an implantable device against a portion of tubular or sac wall of said vessel at said site, whereby load applied to said vessel is borne by said wall and said device, said vessel being assisted by said device when said wall and said device acts upon said load, said device including an energy storage means which is charged with a pressure or energy charge by means of said load being applied to said vessel, said device including a cuff containing an adjustable cuff tensioner, and or an adjustable outer cushion to protect surrounding vessels, and tissues, said cuff shaped to secure around and along a vessel.

The present invention also provides a method of treating a vessel in a human or animal body, said method including the steps of: preparing a patient; identifying a site in said vessel requiring treatment; replacing all or a portion of said vessel requiring treatment with an implantable device, whereby load applied to the vessel is borne by said device, said vessel being assisted by said device when said device acts upon said load, said device including an energy storage means which is charged with a pressure or energy charge by means of said load being applied to said vessel, the method including application of a cuff containing an adjustable cuff tensioner, and or an adjustable outer cushion to protect surrounding vessels and tissues.

The pressure or energy charge can be an energy charge which is at least in part produced by elastic deformation of said device.

Operation of said device can result in a system containing said vessel operating in a less stiff and or more compliant manner than would have been present from said portion of said wall at said site as untreated.

The energy storage means releases said pressure or energy charge to enable said device to assist said wall when said wall acts upon said load.

The device can include at least one elastomeric component, said elastomeric component being adapted to release energy to assist said vessel.

The device or said energy storage means releases said pressure or energy charge in response to unloading of said vessel.

The method can include positioning a cuff, which is a part of said device, around said wall.

The cuff can contain a tensioner to adjust the device coupling with the vessel.

The cuff can contain a cut out window section allowing for increased energy and volume change.

The cuff can contain an elastomeric window in the cut out without additional components or in addition to the changeable volume portion.

The cuff can contain cut out sections to allow for a smaller radius of curvature such as that on the inner radius of the ascending aorta.

The cuff can be bonded to the changeable volume portion or energy storage device using heat or a biocompatible glue treatment.

The cuff can be fixed to the changeable volume portion or energy storage device using additional cuff flaps which attach to the sides of said device.

The cuff can be attached to the changeable volume portion or energy storage device via one wire or two wire members running along the length of the device.

The energy storage means can be a windkessel or deformable reservoir

The energy storage means can include a compressible media chamber which when compressed stores said pressure or energy charge.

The energy storage means can include an electronic energy harvesting means.

The compliance of the device can be modified at the time of implant by inflation and or after implantation.

The compliance of the device can be modified after implant by using a subcutaneous port under the skin, which is attached to the changeable volume portion and energy storage device via a connected tube. A subcutaneous needle is inserted through the skin to the implant port to add or remove volume.

The connected tubing may be wire reinforced and may include multiple lumens allowing for one or more volume and energy connections, or independent tensioning, and may include insulated electrical conductors to power and receive data from attached sensors.

The performance of the device can be monitored by an electronic sensor mounted in the subcutaneous port and or mounted in the changeable volume portion and energy storage device.

The sensor can be powered by an attached implanted battery, an attached implanted induction coil charged via inductive power delivered by an external coil, or via electrical power connected when an electrical subcutaneous port and power needle are used.

The sensor can be connected to an electronic communications circuit via analogue to digital conversion or via a digital connection. The electronic communication circuit can send data electronically via RF, wi-fi, or blue tooth to an external receiver to log and record data.

Compliance can be modified by inflation with one, or a combination of more than one, of the following media: a bio-compatible fluid; liquid silicone; liquid saline; a liquid containing a contrast agent (x-ray viewable); a gel solution that expands with temperature to a final operating volume at 37° degrees Celsius; uncured or liquid polymer which is thermosetting, at 37° C. or via activation by light or heat; a heat activated gel; elastin; collagen; elastin and collagen in combination; air; a polymer that cures or thermosets after injecting. gas, carbon dioxide, helium, or air or other compressible media, water.

The vessel can be a blood vessel.

The load applied to said vessel being borne by said wall and said device can be a systole phase of a cardiovascular system.

When said wall and said device acts upon said load, it can be a diastole phase of a cardiovascular system.

The device can be positioned externally onto said vessel or the device can be positioned within said vessel or the device can be positioned between cut ends of said vessel to replace said site.

The present invention further provides a treatment or assistance device for operating in or with a tubular or sac wall of a vessel in a human or animal body, said device including a changeable volume portion which is adapted to interact with said vessel so as to modify the volume of said vessel; and an energy storage means functioning with said changeable volume portion whereby a decrease in the volume of said changeable volume portion creates a pressure or energy charge in said energy storage means, said pressure or energy charge being able to be subsequently released to cause said changeable volume portion to increase in volume.

The changeable volume portion can be a cuff member which includes an inflatable portion, said cuff member and said inflatable portion being able to be positioned around said vessel, said cuff member can contain a tensioner to increase or decrease the working range of the changeable volume portion.

The energy storage means can be a pressure storage means such as a windkessel or deformable reservoir, or balloon. The pressure storage means include at least one valve, or at least one respective valve, to control the rate of charging and the rate of discharging of said pressure charge.

The changeable volume portion can be constructed at least in part from an elastomeric material, said elastomeric material being said energy storage means.

The changeable volume portion can be a graft or a stent graft or a part thereof and said energy storage means is an elastomeric material which forms said graft, or said stent, or said part, or a deformable reservoir, or a balloon/s with graft and or stent structures.

The deformable reservoir, balloon, or stent can have multiple elements in series or parallel to improve the overall performance.

The changeable volume portion and said energy storage means can be are primed with a threshold or reference pressure and or volume. The volume and energy of the device can be modified after implant by using a subcutaneous port under the skin, which is attached to the changeable volume portion and energy storage device via a connected tube. A subcutaneous needle is inserted through the skin into the implanted subcutaneous port to add or remove volume.

The media with which the changeable volume portion can be primed with one or more of the following media: a bio-compatible fluid; liquid silicone; liquid saline; a liquid containing a contrast agent which is x-ray viewable; a gel or other solution that expands with temperature to a final operating volume at 37° degrees Celsius; elastin; collagen; elastin and collagen in combination; air; carbon dioxide; helium, or a gas, water, or an incompressible media.

The energy storage means can include a compressible fluid chamber.

Media with which said energy storage means can be primed is one or more of the following compressible media: air; carbon dioxide, helium, or gas, or other compressible media.

The changeable volume portion can include a generally inextensible outer portion whereby any change of volume is confined to being within the volume defined by said outer portion.

The device can be adapted, at least in part, to be implanted into a human or animal body, by being compressed and partially rolled into a tube that can fit into an endoscopy port for minimally invasive surgical (MIS) deployment, or can be implanted using normal sternotomy (open chest) surgery, or a minimal right or left sided thoracotomy between the first or second or another intercoastal spacing (between the ribs).

The device cuff can have an attached tape that facilitates loading into the tube, which can then be used to pull on using endoscopy instruments for unloading and tracking the device around the vessel to position the device on the vessel at the treatment site.

The cuff ends can facilitate clamping using normal or endoscopy clamps, said cuff ends are then sutured, or bonded using an agent that sets at 3 7 C, or using an agent activated using UV light delivered from another endoscopy port, heat welded, or fixed using a mechanical clamp via a second endoscopy port.

The cuff end can be wrapped around the vessel, and can be a single piece for attaching back to the start of the cuff. The cuff ends can be multiple sections to allow for independent tensioning of the device from the proximal end of the vessel to the distal end of the vessel.

The changeable volume portion and said energy storage means can implanted in said human or animal body.

The changeable volume portion can be implanted in said human or animal body while said energy storage means, if separate from said changeable volume portion, can be located outside of said human or animal body.

The changeable volume portion can be joined to ends of said vessel.

The changeable volume portion can be attached externally to said vessel.

The changeable volume portion can be attached in the vessel.

The device can be used to treat or assist a blood carrying vessel.

The device can be used to repair the compliance of a portion of said vessel.

The device can be used to modify the systolic and diastolic characteristics of said vessel to thereby improve cardiovascular performance.

The tensioner allows for an adjustable coupling of the device and vessel by use of another deformable reservoir, stent, graft, or stent graft, and or balloon that can be inflated via its own port.

Around the device, an adjustable outer means formed by another deformable reservoir, stent, graft, stent graft, and or balloon can be used to cushion any contact with surrounding vessels and tissues such as the pulmonary artery, the superior vena cava, and the lung.

The changeable volume portion can include electronic dynamic dampening control and energy harvesting and discharging means.

The device can be applied to the ascending aorta by isolating it from the pulmonary artery.

The device can be applied to both the ascending aorta and the pulmonary artery.

The device can be applied to multiple vessels including the ascending and descending vessels attached to both the right and left sides of the heart.

Load sensors attached by an electronic circuit and a data logger can be applied at time of implant to quantitate the cuff tension and balloon to vessel coupling, allowing adjustment of the cuff ends to balance the load at each side on the device prior to suturing.

Load sensors could remain implanted and used to monitor and track the stability of the device over time.

### BRIEF DESCRIPTION OF THE DRAWINGS

Various embodiments of the present invention will now be described, by way of example only, with reference to the accompanying drawings. The components in the drawings are not necessarily to scale, with emphasis instead being placed upon clearly illustrating the principles of the disclosure.

FIG. 1 is a device prototype prior to attachment with attached subcutaneous port;

FIG. 2 is a device attached and clamped onto a pressurized model aorta;

FIG. 3 is a cuff device on an aorta;

FIG. 4 shows an effect of balloon media and a durometer on aorta stiffness;

FIG. 5 shows an effect of balloon media and a durometer on pulse pressure reduction;

FIG. 6 is a photo sequence showing device partially rolled and inserted into an implantation tool;

FIG. 7 is a device unloading and inflated from the implantation tool;

FIG. 8 is a photograph showing a device being unloaded using endoscopy ports in simulated bench procedure;

FIG. 9 is a photograph showing endoscopy ports being inserted in a pig model (endoscopy camera and instrument port);

FIG. 10 is a sequence of photographs showing the device being inserted into the endoscopy port and pulled out within the pig chest using the endoscopy clamp (the endoscopy camera shows the device cuff and clamp on the monitor);

FIG. 11 is a sequence of photographs showing the device cuff manipulated around the aorta by the cuff tag;

FIG. 12 is a sequence of photographs showing the device cuff alignment, clamping and balloon inflation;

FIG. 13 is a sequence of photographs showing a skin pocket being made for the subcutaneous port and being inserted into the pocket;

FIG. 14 is a device drawing showing the changeable volume section and the cuff tensioner;

FIG. 15 is a device drawing showing the changeable volume section and the cuff tensioner around a vessel;

FIG. 16 is a device drawing showing the changeable volume section and the cuff tensioner strapped to a vessel;

FIG. 17 is a device drawing showing changeable volume section with cuff tensioner and outer deformable cushion;

FIG. 18 is a device drawing showing the changeable volume section with attached energy harvesting electronic circuit;

FIG. 19 is a schematic of a normal aorta with a corresponding pressure graph;

FIG. 20 is a schematic of an aged aorta with a corresponding pressure graph;

FIG. 21 is a stress-strain relationship graph for typically used graft materials as well as various blood vessels of the human body;

FIG. 22 illustrates a cross section through an inflatable section;

FIG. 23 illustrates a diagrammatic perspective view of the inflatable section of the intraluminal device of FIG. 23;

FIG. 24 is a schematic representation of an extraluminal or intraluminal inflation system which can be used with a device, where FIGS. 22, 23 and 24 are respectively from PCT/AU2005/000299 and the full text of which is incorporated herein by reference;

FIG. 25 shows a cuff with cut outs for shaping the cuff around the aorta inner radius;

FIG. 26 shows various cuff end flaps configurations for independent tensioning (1 flap, 2 flaps, 3 flaps);

FIG. 27 shows a cuff with a cut out window;

FIG. 28 shows a double bar cuff attachment;

FIG. 29 shows a single bar cuff attachment;

FIG. 30 shows a split cuff single bar attachment;

FIG. 31 shows adhesive used for the cuff attachment;

FIG. 32 shows a cuff attached using side flaps to the balloon;

FIG. 33 shows a wire reinforced inflation tube and a multi-lumen tube for media and electrical connections;

FIG. 34 shows sensors mounted in the subcutaneous port;

FIG. 35 shows a sensor mounted in balloon via the inflation tubing and subcutaneous port;

FIG. 36 shows a sensor powering and monitoring block diagram;

FIG. 37 shows electronic wireless charging and wireless data transmission and reading circuit;

FIG. 38 depicts implanted and external system components;

FIG. 39 shows a subcutaneous port with electrical power needle;

FIG. 40 shows multiple balloons used in parallel;

FIG. 41 shows multiple balloons in series (stacked).

FIG. 42 shows a reduced width cuff in middle of balloon;

FIG. 43 shows separate charge and discharge paths and features;

FIG. 44 shows separate charge and discharge circuits with electronic switching and timing control;

FIG. 45 shows a device containing a co-axial electromagnetically controlled sliding co-axial tube for dynamic dampening and discharge control;

FIG. 46 depicts a cuff window containing an attached deformable sheet; and

FIG. 47 shows a load sensor for balancing cuff load when device is attached to a vessel, the location for isolating the ascending aorta and pulmonary artery, the cuff insertion and cuff suturing location, and devices used on both the ascending aorta and pulmonary artery.

### DETAILED DESCRIPTION

Illustrated in FIG. 21 is a stress-strain relationship graph indicating typically used graft materials and a comparison against normal blood vessels of the human body. The values plotted for the materials clearly indicate that they are not compliant enough for reducing stiffness in vessel wall applications. Age or otherwise stiffened vessels have a stress-strain relationship equivalent to PET and PTFE.

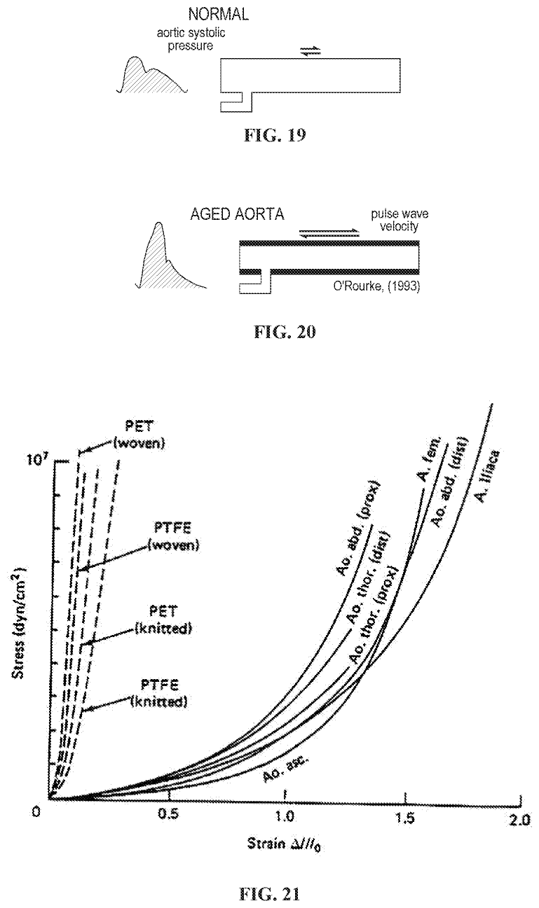

The increased stiffness of aged vessels, results in a greater aortic systolic pressure and a reduced pressure decay during diastole, than compared to younger vessels, as indicated in FIGS. 19 and 20.

Aneurysm treatment using stent grafts suffer from leakage, migration and can leave a significant unfilled zone between the aneurysms sac and the stent graft, and additionally they reduce arterial compliance by use of non-compliant materials shown to increase systolic pressure and lower diastolic discharge much like an aged stiffened vessel.

It is to these difficulties that the following described embodiments are addressed in order to attempt to alleviate or ameliorate one or more of these difficulties.

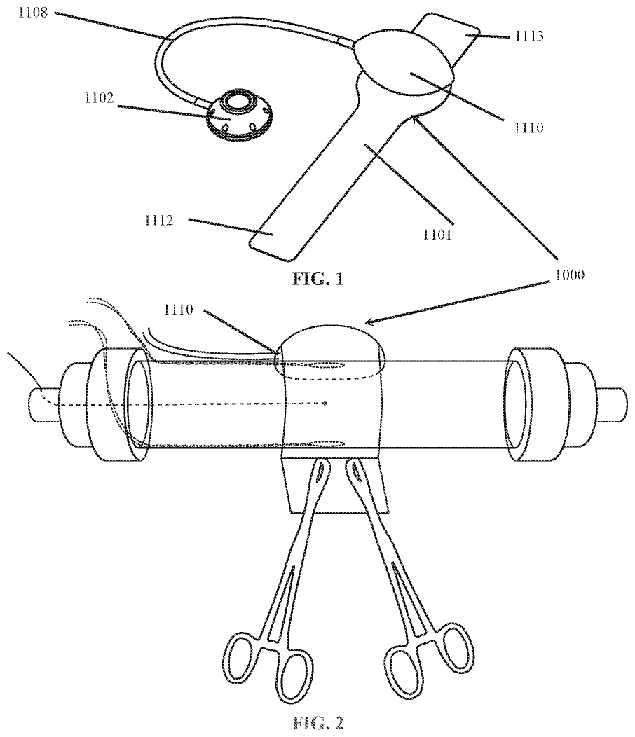

Extraluminal Cuff 1101 With Inflatable Cuff Balloon 1110 Passive Recoil Inflatable Cuff Balloon. Illustrated in FIG. 1 is a device 1000 and includes a flexible cuff 1101 which can be positioned around a vessel or conduit, which can be the ascending aorta, but can be any aortic vessel attached to the left side of the heart, the pulmonary artery or any vessel on the right side of the heart and circulatory system or other vessel, or body conduit. The cuff 1101 includes an inflatable portion or bag referred to as the cuff balloon 1110.

The cuff 1101 includes a subcutaneous port 1102 having a septum seal, allowing the cuff balloon 1101 to be filled at the time of implantation., or adjusted after implantation by means external to the body, such as via a syringe and needle access through the chest wall. The cuff 1101 can be implanted thorascopically.

The cuff balloon 1110 is flexible along its width and length and is contained circumferentially by the cuff when the balloon is pressurized thus, allowing an efficient coupling between the cuff balloon 1110 and the outer wall of the vessel 1100. This is shown in FIG. 2 where the device is clamped to a pressurized model of a stiffened aorta.

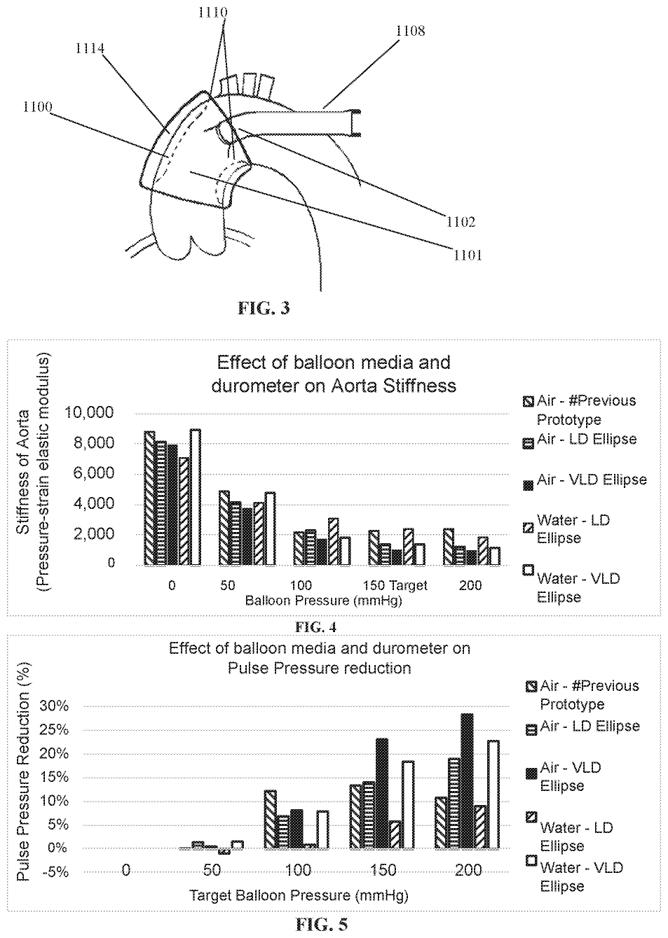

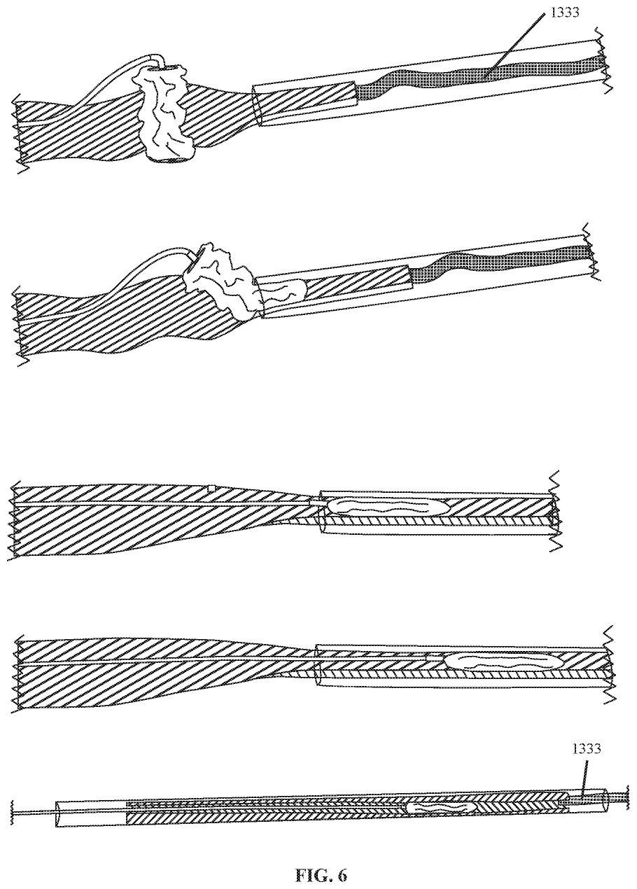

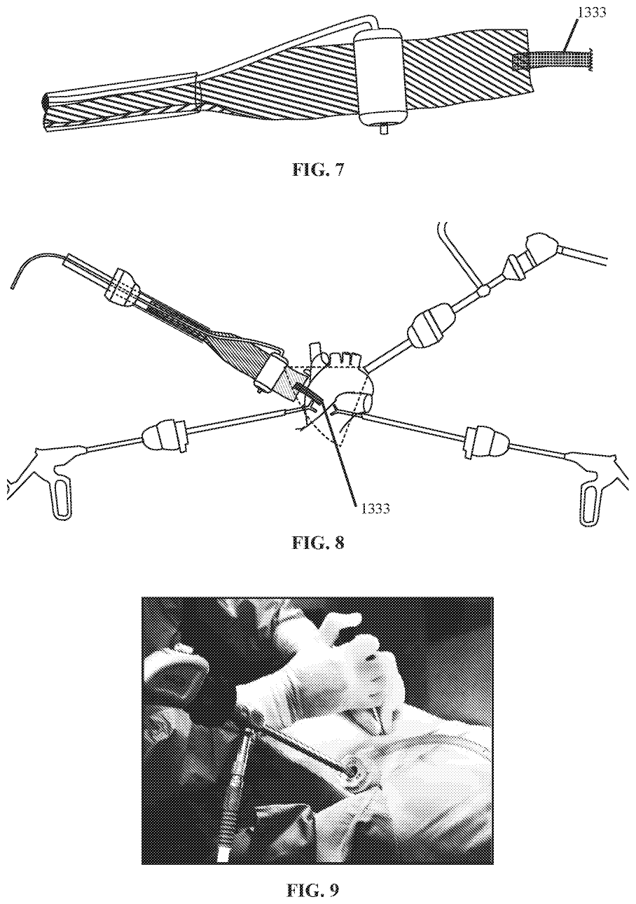

As shown in FIG. 3, the cuff 1101 can include reinforcing fibers or wire struts (not illustrated) which are affixed to or are imbedded in, the outer layer 1114 in order to maximize transfer of loads between the cuff balloon 1110 and the wall of the vessel 1100. The cuff 1101 is flexible enough to follow the shape of the outer wall of the vessel 1100 when applied. Once the cuff 1101 is positioned around the outer wall of the vessel 1100, the opposed ends 1112 and 1113 will be adjacent to each other, where they are overlapped and locked together via any appropriate means such as staples, pegs, suturing, gluing etc., so that the cuff 1101 can remain in place surrounding the vessel 1100.

The reinforcing fibers or wire struts (not illustrated), allow the cuff 1101 to maintain its flexibility so as to be positioned around the outside of the wall of the vessel 1100, but also allow the outer surfaces of the cuff 1101 to be relatively inextensible, whereby the change in volume of the cuff balloon 1110 is transmitted to compress or allow expansion of the wall of the vessel 1100.

The cuff 1101 is intended to sit gently against and around the outer wall of the vessel with the cuff balloon 1110 reducing the vessel diameter by 1% to 50% at a set threshold cuff inflation pressure). The reduction can be greater depending on the conditions of the patients and the properties of the vessel wall.

The cuff 1101 can be made of an implantable graft material such as PET polyurethane, silicone, a combination of polyurethane and silicone, or other biocompatible polymeric material, or fiber-reinforced biocompatible polymeric materials. The cuff balloon 1110 can be made of flexible polyurethane, silicone, a combination of polyurethane and silicone, or other polymeric material, or elastomeric polyurethane, silicone, a combination of polyurethane and silicone, or other polymeric materials

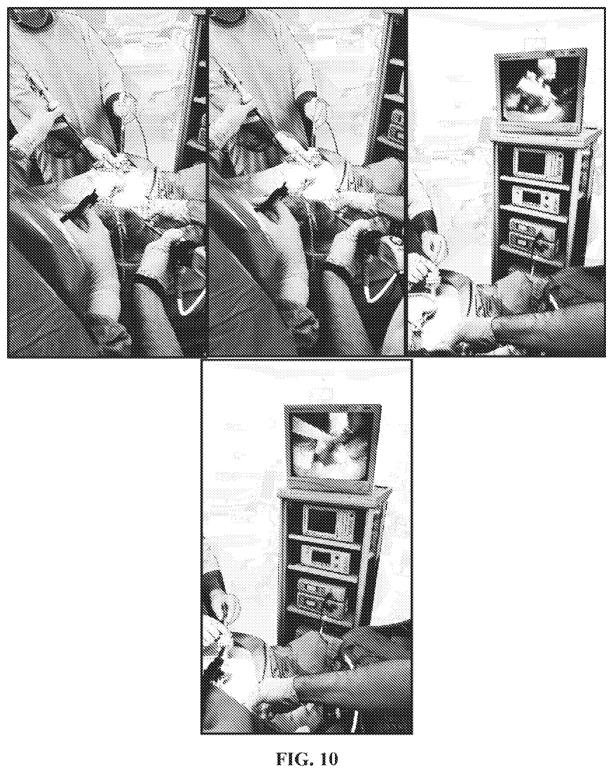

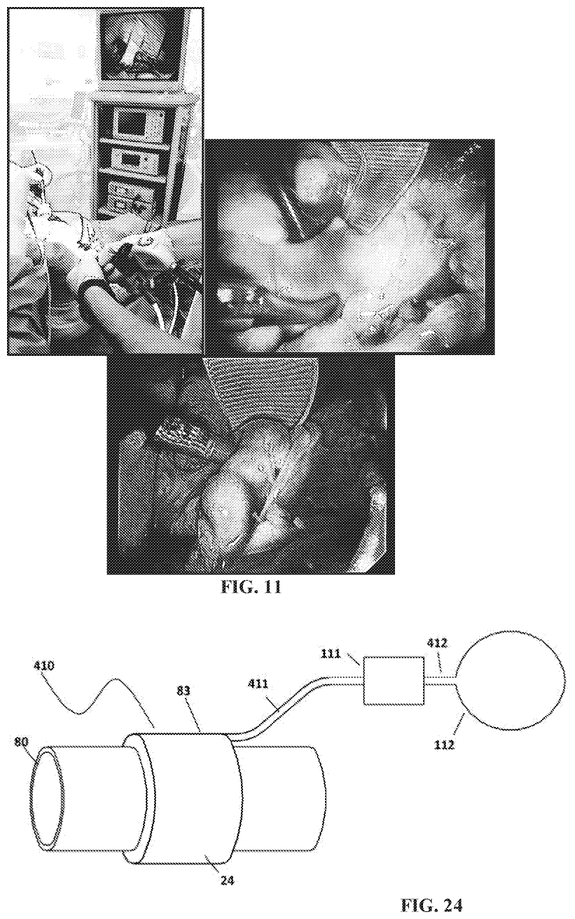

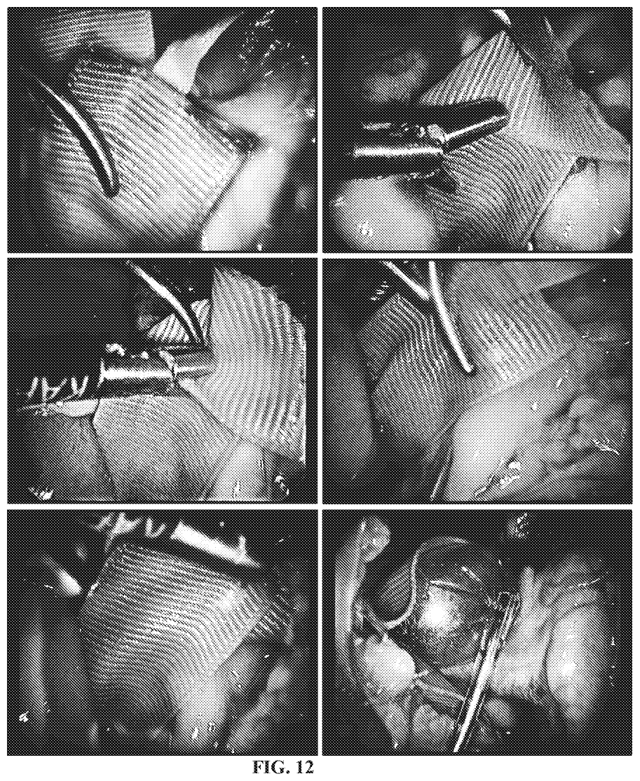

The above device is flexible and compressible enough so that it can be partially rolled across its width and inserted into a deployment tool (tube) along the device's length. This is shown FIG. 6. The cuff has an attached tag or tape 1333 (thinner) that allows for loading and can be used to unload as described below. Unloading the device from the deployment tool is shown in FIG. 7. The deployment tool can fit into a standard endoscopy TROCAR port and allows for a surgical instrument to access the tag or tape 1333 via an additional endoscopy port, to pull the device out of the tube as demonstrated in bench tests in FIG. 8. The endoscopy ports can be inserted in the intercostal space (between ribs) as indicated in a pig animal model in FIG. 9. The device can then be loaded through the port and deployed with an endoscopy instrument as shown in FIG. 10. The cuff tag (or cuff tape) attached to the cuff is used to facilitate removal of the device within the chest adjacent to the aorta, and the tape is used to manipulate the cuff around the aorta into position (as shown in FIGS. 11 and 12).

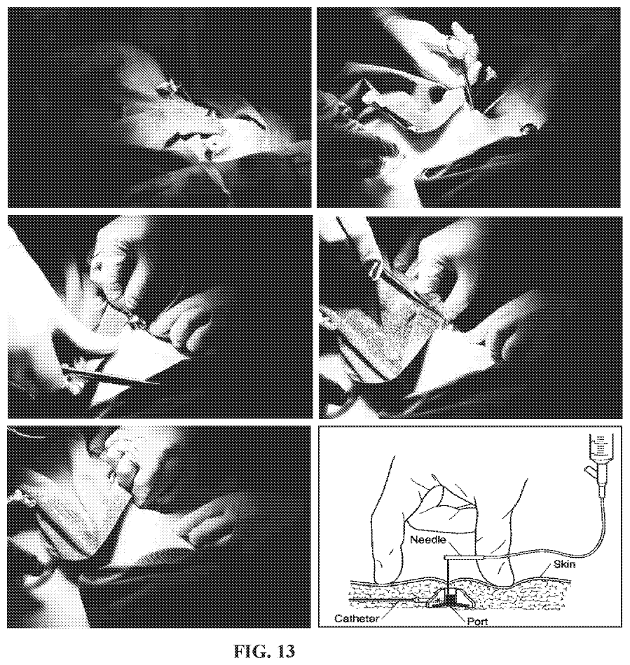

The tubing connected to the cuff balloon runs out of the deployment tool through the port. A skin pocket can then be made adjacent to the port hole where the subcutaneous port is inserted and attached to the tubing as shown in FIG. 13.

Passive Recoil Inflatable Cuff Balloon with Cuff Tensioner. An inflatable cuff tensioner (FIG. 14) can be used to further control the cuff balloon to vessel coupling. This is useful as it allows the cuff ends to be fixed into position with the tensioner inflated partially, thereafter allowing deflation or further inflation after the cuff balloon is inflated. A greater range of change of volume between the cuff balloon and vessel is achievable while maintaining a cuff balloon inflation pressure that is optimal for a specific patient and can be tuned post deployment in the clinic. One of the balloons is used for normal compliance, and the other balloon is used to tighten or loosen the compliance balloon against the vessel, this balloon may be made of a stiffer material and or is filled with media at a higher pressure or as needed.

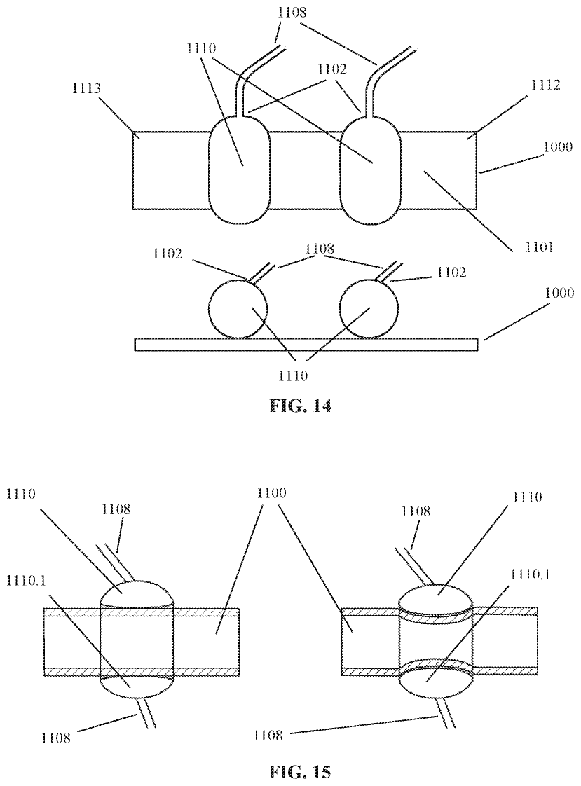

FIG. 15 (left side) shows a cuff tensioner (lower balloon) 1110.1 on a vessel 1100 with less volume than the compliant cuff balloon (top balloon) 1110. When the cuff tensioner is inflated (right side image of FIG. 15), the compliant balloon is pulled tight against the vessel facilitating more change of volume between the two, and therefore adding more compliance to the vessel during systole (expansion), and more counterpulsating (compression) during diastole.

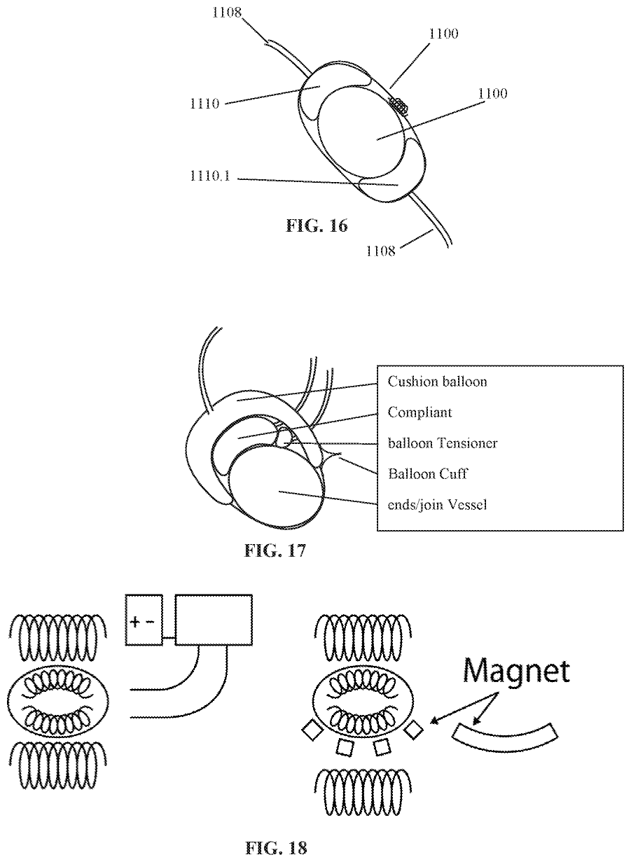

Each balloon 1110 and 1110.1 can have separate inflation lines 1108 as indicated in FIGS. 15 and 16, the latter showing the cuff 1101 attached around a vessel 1100 in cross section. The tubing could be two separate lines to two separate ports or a tube with multiple lumens could be used to one port and a multi luminal needle used to connect each ort to separate external media and pressures levels.

These balloons may be formed using multiple balloons for function as a compliant balloon or a tensioner balloon.

Passive Recoil Inflatable Cuff Balloon with Cuff Tensioner and Outer Cushion. Further, an additional balloon could be added to the outer surface of the cuff, shaped and positioned to cushion the surrounding vessels and tissues as shown in FIG. 17 (3 balloons in total each with its own inflation port). This could help the device interaction by not disturbing the pulmonary artery, the superior vena cava, the lung, and could also be formed of separate balloons or multiple balloons for each contact area. This outer balloon could be adjusted after implant to offer the best cushion conditions and may also be a useful barrier to fibrous tissue encapsulation known to grow around implanted materials. Limiting or controlling the fibrous tissue growth may be achieved by expanding the cushion volume during the healing phase post implantation, and reducing the cushion volume if a thicker more mature fibrous growth needs to be reduced. Additionally, the cushion balloon may be filled with bio active agent/s that secrete out of this balloon by membrane osmosis, or by slow discharge along the connecting port line or via a dedicated release port. The cushion, tensioner, cuff balloon, cuff, or connection port and tubes, could be partially and completely coated in a non-stick anti-inflammatory agents to inhibit undesired localized tissue responses around the implant.

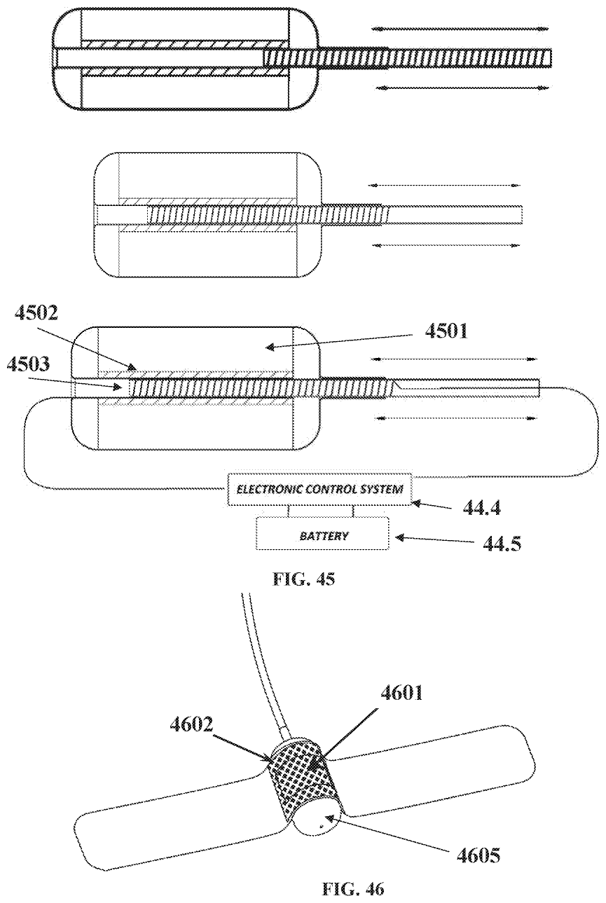

Inflatable Cuff Balloon with Electronic Energy Harvesting. Shown in FIG. 18 is a device drawing showing the changeable volume section with attached energy harvesting electronic circuit. The balloon has an electromagnetic coil attached to either side which when deformed, charges an attached battery. Magnetic components could also be used within the balloon, as a coating, or as particles in the balloon media to establish a suitable magnetic field for improved energy harvesting. A control system could decide if extra energy is needed during diastole to actively deform the balloon and therefore the aorta for added performance, and if desired, dynamic damping and dynamic discharge can be used to control the speed of the dampening phase in systole, and the speed of energy discharge in diastole, which may involve the need to electronic power to be supplied dynamically to the electromagnetic coils. A similar electronic system is referred to in FIG. 45. The above devices could be in part intraluminal and could be formed by flexible stents in addition to balloons, membranes, deformable reservoirs, air chambers, bellows, or Windkessells.

Additional Features. A windkessel can be connected to the inflation lines 1108, as described in respect of FIG. 1 of PCT/AU2005/000299 (windkessel is labelled 1125 in FIG. 1), invented by the current inventor and published in 2005, which is incorporated herein by reference. However, additional to a windkessel could be a system to increase or decrease the bias pressure. Such a system could comprise a syringe piston where the piston is incrementally stepped to increase or decrease the pressure in the windkessel gas chamber (1104 in Figure! of PCT/AU2005/000299) to a set mean operational level. The system could have a micro stepper motor that can be locked into position when set thus only requiring power when the motor is moved. Appropriate control electronics would need to be incorporated which could be battery powered and consist of an electronic sensor to activate changes in response to an externally triggered coded electronic signal.

A second windkessel with a vacuum bias could be used in conjunction with the windkessel 1125 (in FIG. 1 of PCT/AU2005/000299) with a positive pressure bias, and be controlled to switch between each, gated by ECG or blood pressure, to act as a pump. Increased cuff operating pressures can then be achievable by increasing each windkessel bias pressure, the positive and negative (vacuum) pressures. Such a system would need volume control (flow per time) measurement in conjunction with the switching control, to maintain the transfer volumes and operating state of each windkessel.

Such a pump system could be configured to control ventricular wall movement to enhance ventricular performance by extra-ventricular compression using external ventricular cuffs. The pump could also be used to inflate an intra-ventricular balloon for blood displacement via a transventricular connection through the ventricle wall.

If so desired, the Windkessel could also be driven by a pump system directly via port 1105 (in FIG. 1 of PCT/AU2005/000299) or by replacing the Windkessel housing to drive the diaphragm directly. This could be used if a patient's heart failure progresses at some future time, such a system being applied as an upgrade and making use of previously installed components.

In its simplest form, the windkessel system of FIG. 1 once adjusted, does not require a pump or electrical power to be operated. The electronic add-on systems described above while adding extensions to the system, require only small amounts of power easily delivered over many years of operation from an internal battery source, having an operating life much like an implantable pacer or defibrillator system.

The system is a simple low cost alternative to the high cost more complex extra-aortic counter-pulsation systems and ventricular assist devices on the marker or being developed for market.

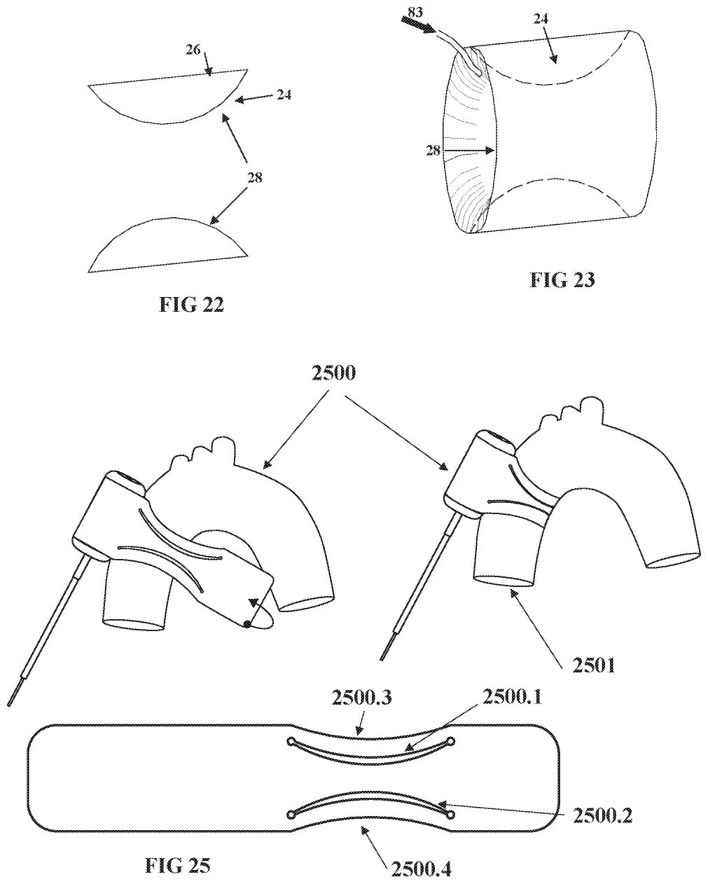

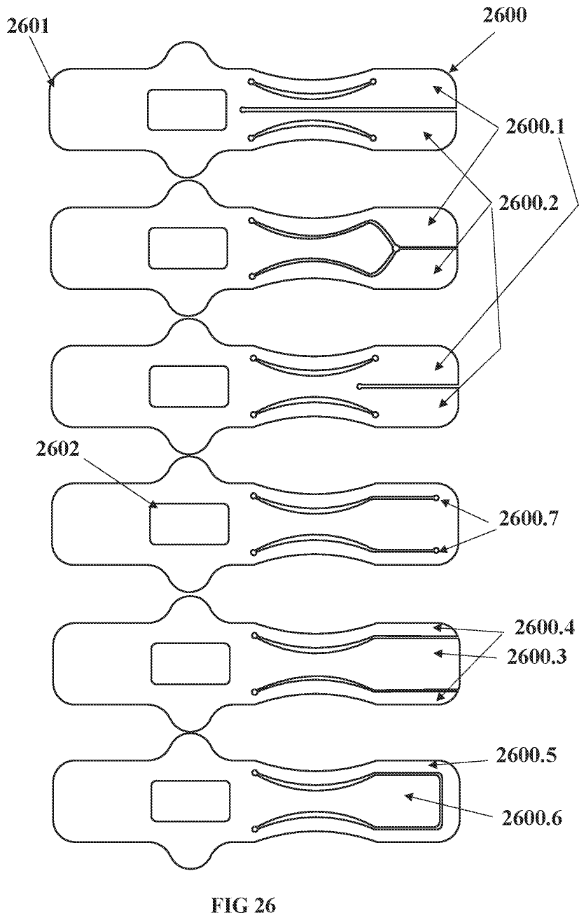

Active Inflation Control System 410. The compliant inflatable pillow 24 of FIGS. 22 and 23 (being FIGS. 13 and 14 from PCT/AU2005/000299) can be such that pressure in the system is preset via the port so as to provide a passive control system. However, in FIG. 24, (being FIG. 19 from PCT/AU2005/000299) there is illustrated a system 410 which includes a compliant inflatable pillow 24 sutured into place as a union between the cut ends of a blood vessel 80, with the section of decreased compliance having been removed. Alternatively, the compliant inflatable pillow 24 could be deployed intraluminally or extraluminally depending upon need.

The system 410 also includes a valve 111 and a diaphragm 112 and a conduit 411 and 412, linking the port 83, the valve 111 and the diaphragm 112 so as to provide active control whereby the pressure strain elastic modulus EP of the compliant inflatable pillow 24 can be adjusted to optimum, or as required.

Such a system may operate after adjustment of the valve, possibly a 2-way valve. Electronic valve control could also be used by including an internal pressure sensor within the pillow or the inflation line leading to the pillow. The measured compliant inflatable pillow 24 pressure would then activate the appropriate valve control using electronic means. More advanced control may be achieved with advanced electronics or a CPU to automate the adjustment process in response to sensed environmental characteristics, such as body temperature, heart rate, blood pressure and other bodily characteristics.

Valve control could allow for different elastic properties between the “charge” (systolic phase) and “discharge” (diastolic phase) phases of the cardiac cycle. This will allow a visco-elastic response that closer resembles the native healthy aorta to be achieved. While the above description is directed to the use of the devices 10, 110, 210, and 310 (see description and drawings of PCT/AU2005/000299) in respect of arteries, it will be readily understood that the embodiments of the invention could be used with veins, and any other tubular walls such as the urethra, or intestines 85.

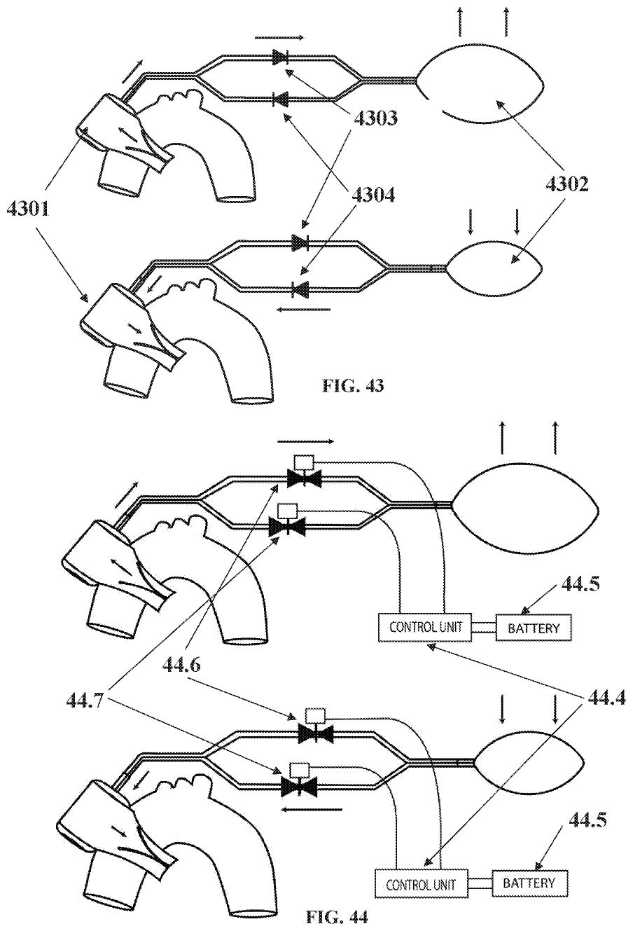

A mechanical means of independently controlling the charge and discharge phases in shown in FIG. 43, which has the changeable volume portion 44re4fjuki/.0 attached to a charge valve and tubing 44.1 to the energy storage device 44.2 (a balloon device in this case), and a separate return path using a discharge valve and tubing 44.3 back to the changeable volume portion. Valve opening and closing for 44.1 and 44.3 can be mechanically set, or sensors 44.6 and 44.7 can monitor their status via electronic switching control 44.4 powered by a battery 44.5 all another form of power delivery as shown in FIGS. 36, 37, 38, and 39.

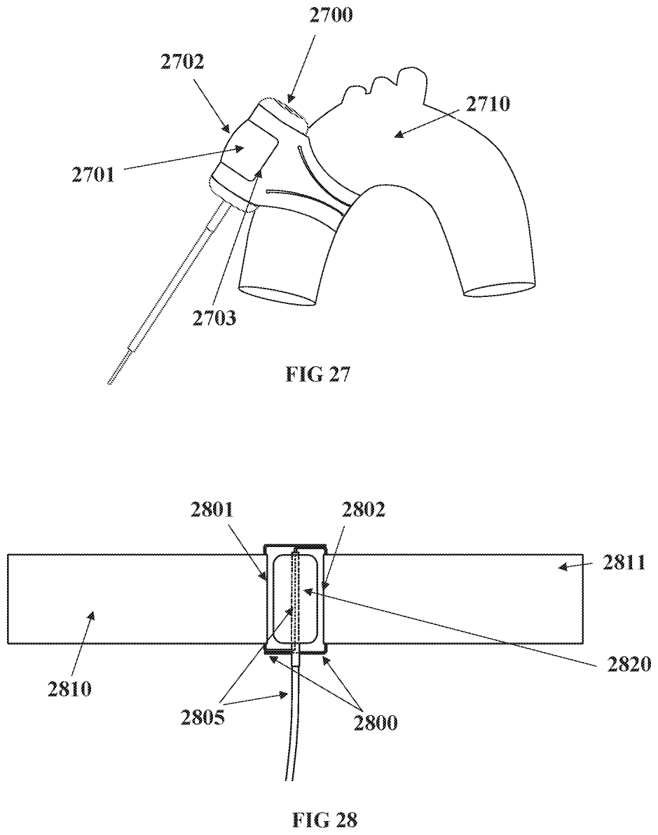

Additional Cuff Features 2500 2600 2700. The cuff can have cut out sections removed (2500.1, 2500.2, 2500.3, 2500,4) as shown in FIG. 25, to allow the cuff to engage a curved vessel such as the ascending aorta inner radius 2501. The cuff can also have separate flaps 2600.1, 2600.2, 2600.3, 2600.4, 2600.5, 2600.6) on the distal end to allow for independent tensioning when fixing the cuff around the vessel back onto the proximal side of the cuff (2601). The flaps may be still attached to each other for sliding the distal cuff end around the vessel 2600.7, which can be cut once in position around the vessel if adjustment of the tension on each side of the device is required. Also shown is a cuff window 2602, where the cuff is removed which allows for greater flexibility of the changeable volume portion balloon 2701 (FIG. 27), as shown in 2700 indicated by the expanded surface 2702 of the balloon 2701, in the cuff window 2703, when the device is attached to the aorta 2710.

Additional Cuff Attachment Features. FIG. 28 shows a double bar cuff attachment 2800, where 2 cuffs 2810 and 2811 are attached to wire bars 2801 and 2802 which are assembled through balloon 2820 in the co-axial fill tubing 2805 and bent around the balloon 2 form the wire bars to attach cuffs 2810 and 2811.

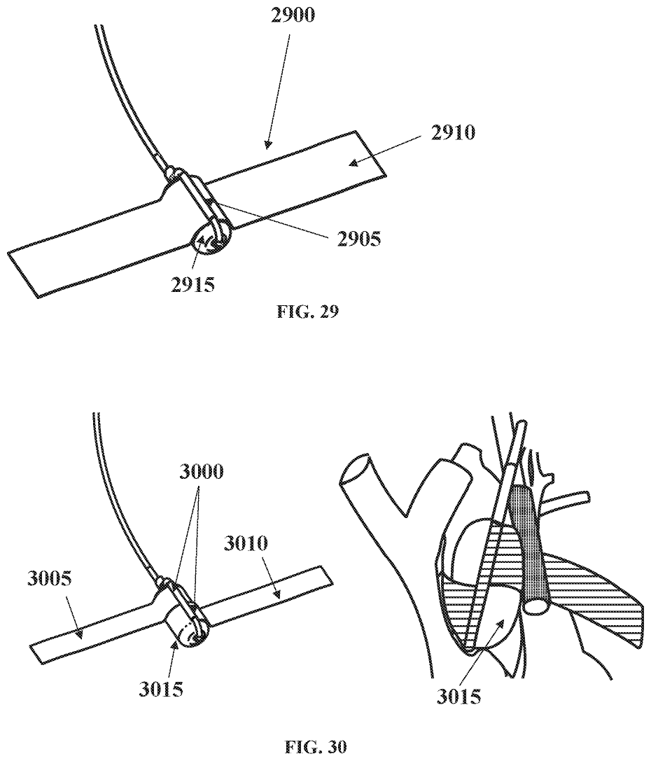

Similarly, in FIG. 29 a single bar cuff attachment 2900 is used once the bar 2905 is inserted through the balloon 2915, allowing cuff 2910 to be attached to the single wire bar.

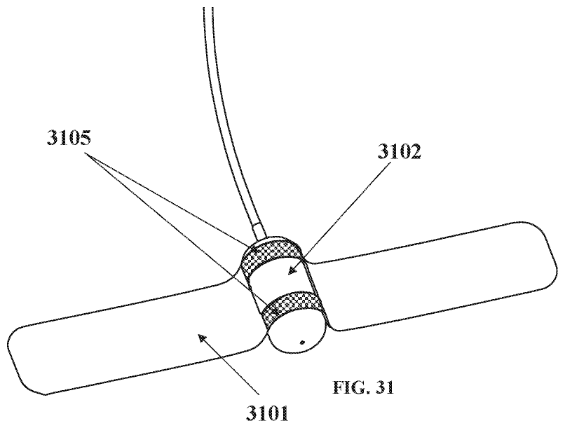

In FIG. 30, split cuff single bar attachment 3000 is shown where the cuff flaps 3005 and 3010 are approximately half the balloon width, which can allow for the flaps to align once the flaps are fixed around a curved vessel such as the ascending aorta. The cuff 3101 can also be bonded 3105 directly to the balloon 3102 as shown in FIG. 31. Bonding 3105 can be achieved by heat bonding the surfaces or using a medical grade adhesive.

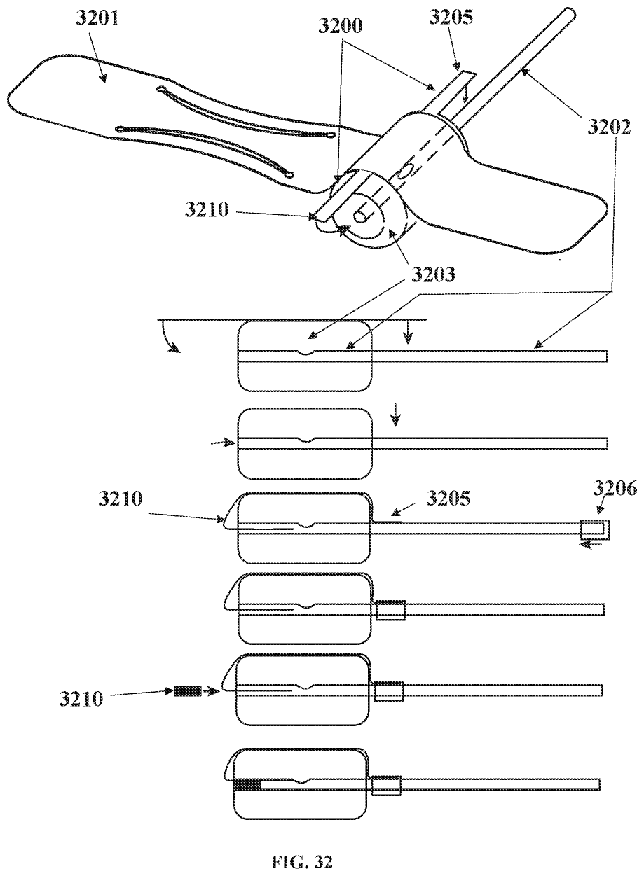

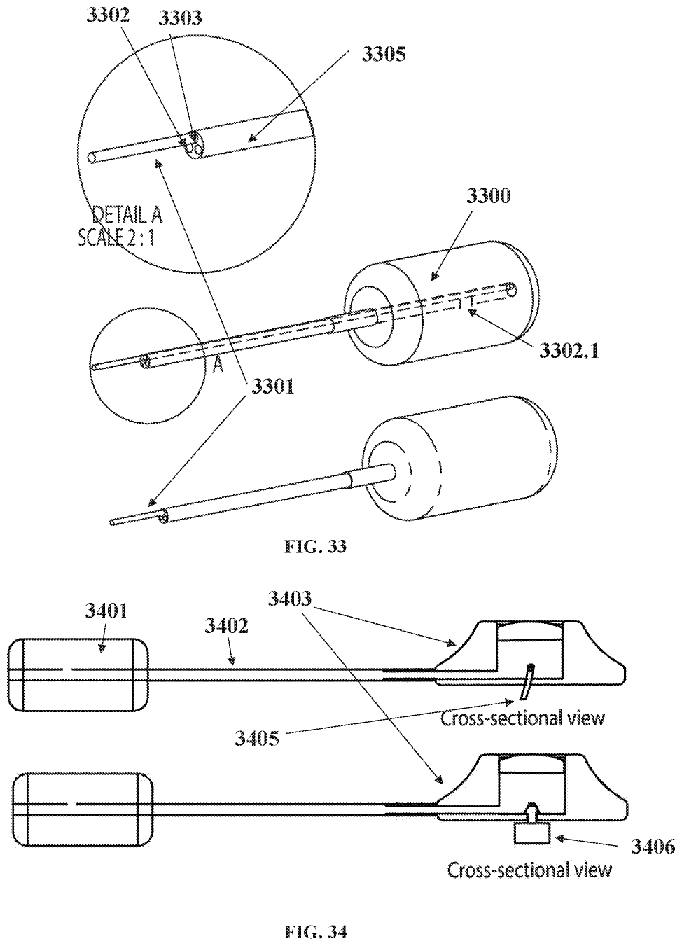

The cuff can be attached to the balloon using side flaps as indicated in FIG. 32. Cuff 3201 has side flaps 3205 and 3210 shaped and sized to attach to the co-axial inflation tubing 3202 on each side of the balloon 3203. The flap 3205 on the proximal end of the tubing, is held onto the outer tube surface with an outer sleeve 3206, all of which can be heat bonded or bonded with adhesive. The distal end flap 3210, is inserted into the inverted distal balloon port thus allowing no protrusion, and once the flap is inserted, a tube plug 3211 to inserted into the lumen which is heat bonded or bonded with adhesive. As shown in FIG. 33, the inflation line 3305 connected to balloon 3300, may have multi lumens. In this case 3301 may be a reinforcement wire to prevent the tube kinking once implanted, 3302 is the media inflation line running into the balloon with internal port 3302.1 to allow for media to enter the balloon. A third lumen 3303 can be used for electrical connections for sensors used with the balloon and attached device as indicated in FIG. 34 and FIG. 48 (load balancing circuit).

Sensor & Electronic Features. FIG. 34 shows a sensor 3405 or 3406 mounted in a subcutaneous port 3403, connected to the balloon 3401 via the inflation tubing 3402. The sensor can be an electronic sensor in a standard electronic package 3406 mounted into the bottom of the port, or can be a MEMs type sensor 3405 on the end of a catheter body with a smaller diameter also mounted into the bottom of the port.

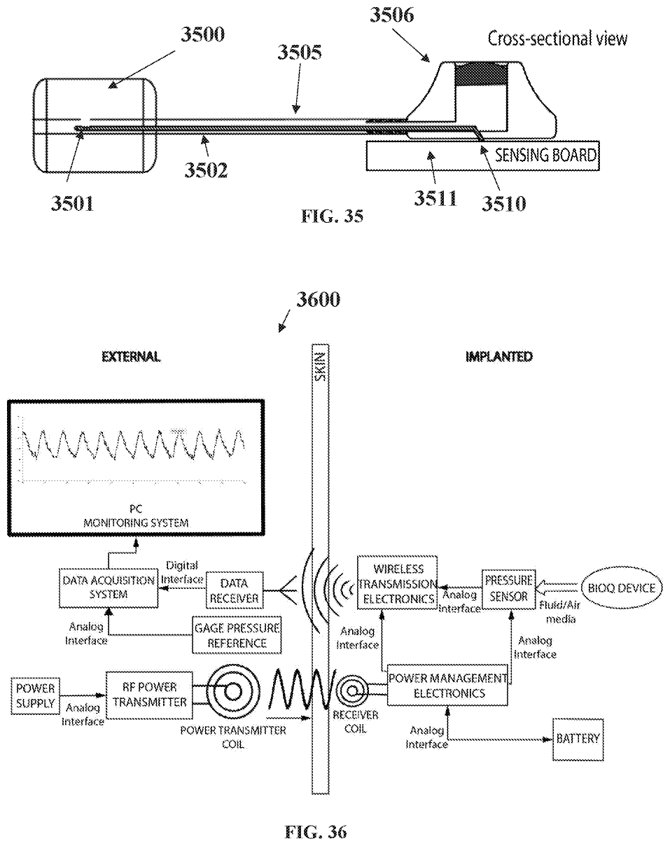

FIG. 35 shows a sensor 3501 in a catheter body 3502 mounted in balloon 3501 via the inflation tubing 3505, attached to the subcutaneous port 3506. The catheter connection is by the bottom of the port 3510 which can be connected to an electronic circuit 3511 for measuring the sensor.

Shown in FIG. 36 is a block diagram 3600 with a sensor measurement, data transmission to an external receiver and data logger, and power transmitter and receiver and battery system. The external instrumentation may include a wireless charging circuit to charge an implanted battery via an inductive coil. Once power is received into the implanted system from the wireless power transmission system, an implanted battery or via the subcutaneous power needle 3901 (FIG. 39), the absolute pressure sensor and the RN4020 Bluetooth module are powered up. This input voltage needs to be regulated due to the low power input needed for the Bluetooth® module and the highly stable voltage needed to ensure the accurate performance of the pressure sensor. Once powered, the sensor measures the absolute pressure inside the balloon or port and generates an analog signal proportional to the pressure level. The on-board analog to digital converter on the RN4020 IC converts this analog signal into digital which is then transmitted using Bluetooth® Low Energy (BLE) technology. On the outside, an external or inbuilt BLE receiver system receives the wireless transmitted data into the computer. An external barometric pressure sensor is read into the computer by the aid of an analog to digital converter and is used to reference the received pressure data to the atmospheric pressure. All the pressure data is managed by software and a gage pressure waveform is finally displayed.

The resonant inductive wireless power transmitter starts with a DC power supply; this DC signal, with a potential Via, is then transformed into AC by the DC/AC Inverter. The inverter consists of switches that are controlled by a micro-controller or a Field Programmable Gate Array (FPGA). By opening and closing alternate switches at a certain frequency fs, square pulses with a magnitude from 0 to Vin volts are generated. These square waves have the same frequency as the switching frequency fs used for the control logic. The square waves coming from the inverter are then transformed into sine waves by the LC resonant circuit that consists of a coil of wire and a capacitor. The LC resonant circuit is tuned to the switching frequency fs in order to maximize power transfer. These sinusoidal waves are then transmitted across the skin to be picked up by the receiver circuitry.

The complete power transmitter circuit is external to the patient and the output power and range depend on the DC supply potential and, coil separation and alignment with the receiver coil.

Implanted in the patient, the power receiver coil inductively picks up the sinusoidal signals coming from the transmitter coil. The LC receiving circuit is closely tuned to the same frequency as the transmitter to maximize the signal pick up. The received sinusoidal signal is then converted to DC by the AC/DC converter, mainly consisting of a diode bridge. This DC voltage can then be used to charge an implanted battery with the aid of the charging control circuitry or can be directly connected to a voltage regulator to ensure a constant voltage potential is sent to the pressure sensing system to ensure optimal operation. The overall efficiency of the wireless energy transmission system will highly depend on how closely matched the transmitter and receiving coils are in terms of resonant frequency. The amount of energy received inside the body will also depend on the distance and alignment between the two coils.

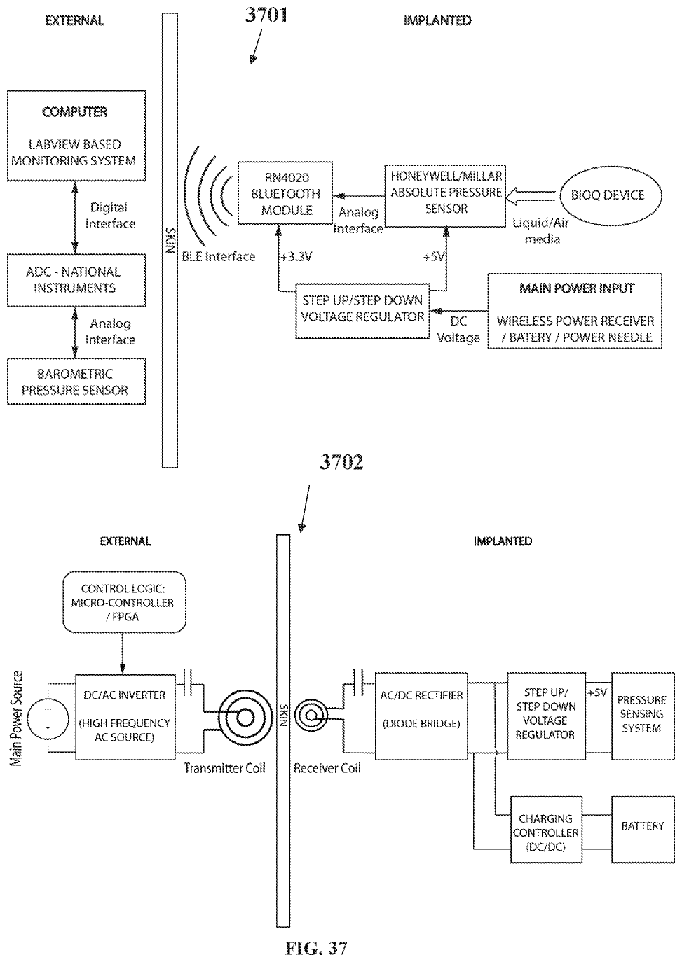

FIG. 37 shows circuit schematics of prototyped functional electronics for sensor measurement and wireless data monitoring 3701 and a wireless power charging and receiver 3702.

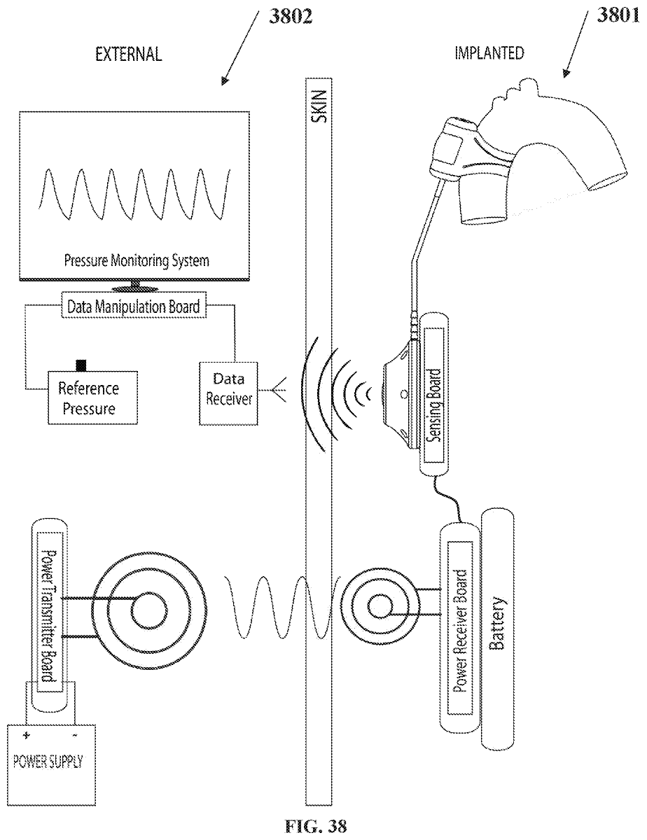

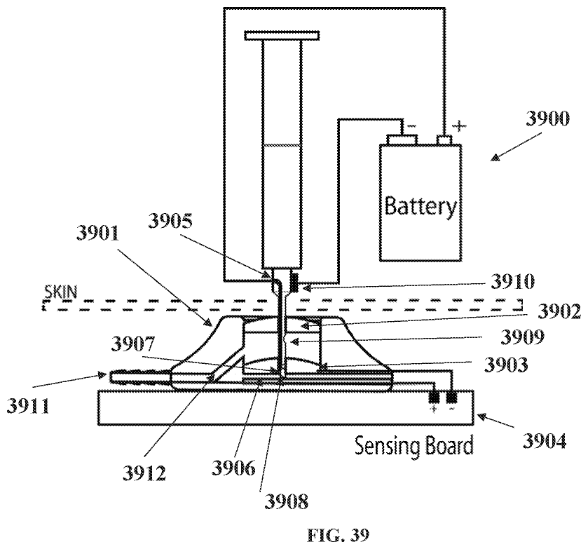

FIG. 38 shows the implanted electronics 3801 and external components 3802 to give the size and locations of the components.

Shown in FIG. 39 is a subcutaneous 3901 which has features to provide electrical power to the implanted electronics. The needle has a ground connection 3910 which is pushed through the port outer sealing septum 3902, which then contacts an inner ground plate septum 3903 which has a conductive coating or imbedded conductive media or nana particles, which is then electrically wired to the negative ground terminal of the attached electronic board 3904. The positive power conductor is an insulated wire feed through needle 3905, which is push through the inner ground plate septum to make contact the positive power plate 3906 at the bottom of the port. The needle tips are sealed and insulated 3907 so it is not able to conduct with the positive plate. The positive power wire is exposed at the tip 3908 to allow conduction with the positive plate 3906. The needle also contains a port 3909 for media to be injected or removed as per normal use of a subcutaneous port which connects the path to the tubing connector 3911 via internal channel 3910.

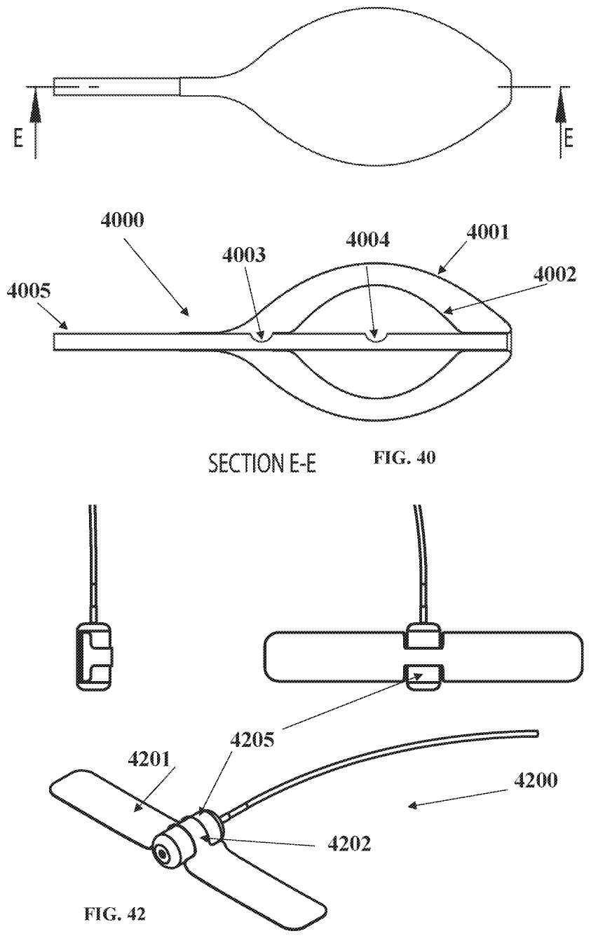

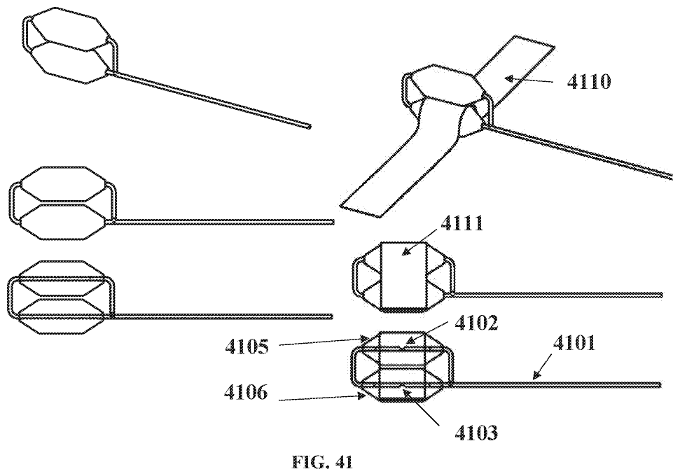

Multiple Balloon Configurations 4000 4100. Shown in FIG. 40 is a multi-balloon configurations 4000 showing two balloons in parallel. Balloon 4001 contains within a second balloon 4002. The media inflation line 4005 connects to balloon 4001 via tube port 4003 and connects to balloon 4002 via tube port 4004. This allows for enhancing the charge and discharge characteristics of the device. The use of balloons in series and stacked vertically on the vessel, as shown in FIG. 41, may also provide desirable charge and discharge characteristics. Inflation tube 4101 connects to balloon 4106 via port 4103 which then is connected to balloon 4105 via port 4102. Cuff 4110 may be attached between the balloons, or may be attached on the top or both balloons 4111.

Narrow Cuff 4200. As shown in FIG. 42, a cuff 4201 has a narrow middle section 4202 which allows balloon 4205 to have more flex at each end.

Additional Charge and Discharge Features. As shown in FIG. 43, a separate charge and separate discharge path can be employed to have greater control of these phases. Balloon (changeable volume portion 4301 to attached to the energy storage device balloon 4302 via tubing and valve 4303 for charging, and discharges via tubing and valve 4304. The valves and tubing length and diameters can be different to enhance the charge and discharge functioning. Further, as shown in FIG. 44, electronic switching and timing control could be added via two sensors 44.6 and 44.7, an electronic sensor control 44.4, and attached power in this case a battery 44.5.

A device containing a co-axial electromagnetically controlled sliding co-axial tube for dynamic dampening and discharge control is also described.

Shown in FIG. 45, the device can contain a co-axial electromagnetically controlled sliding coaxial tube 4502 and 4503, for dynamic dampening and discharge control. The sliding axial tubes allow for the co-axial tube to lengthen in response to the vessel load applied in systole, and shorten to counterpulsate when in diastole. A power source such as a battery 44.5 can be used to power the electromagnet to slow the dampening phase by having more resistance in the co-axial tube to lengthen, or the electromagnet and coil power could be reduced to allow for faster response to dampening. Likewise, during the energy release phase, the co-axial tube shortening could be slowed down by powering the electromagnet to provide more resistance to movement, which could then be activate during the cycle adding more energy to increase the shortening to provide more counterpulsation and energy release. The electromagnet could be used to harvest energy as referenced in FIG. 18, so that the harvested energy is returned in the next phase or cycle, or is used to charge a battery for later use.

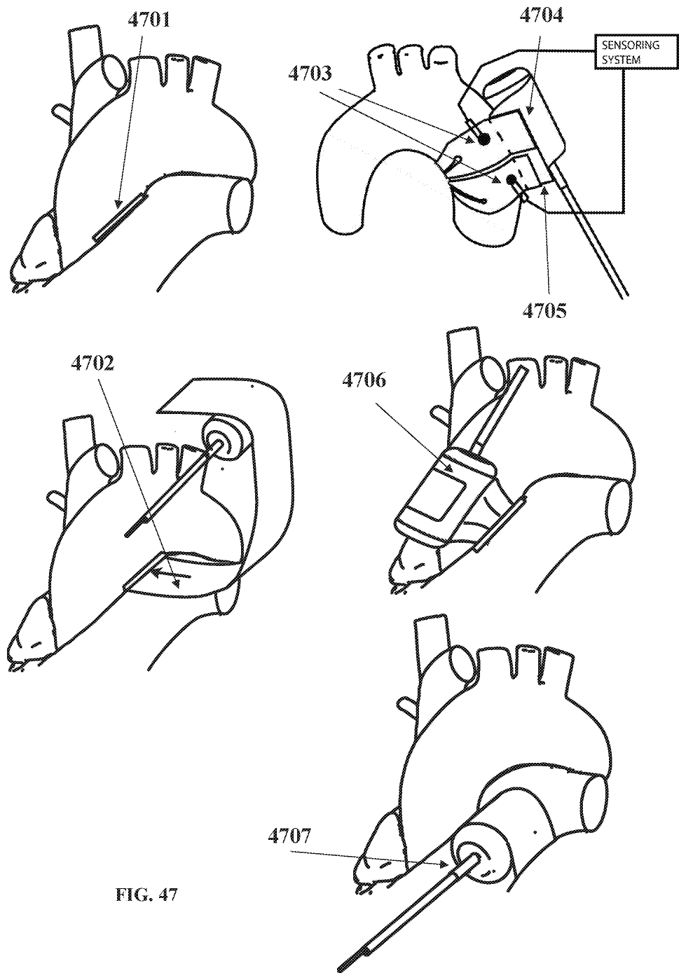

Additional Cuff Window Feature 4601. As shown in FIG. 46, the cuff window 4601 may contain an attached deformable sheet 4602 (deformable window) which is used as an energy storage device in addition to a balloon or other prior referenced means, or could be used alone without another energy storage means. In this case, balloon 4605 is shown which could be used as a changeable volume portion or as a changeable volume portion and an additional energy storage device. The balloon could also be used as a cuff tensioner as prior referenced. Two or more balloons could be used in addition to the deformable window, one as a balloon cuff tensioner, and the other as a changeable volume portion or as a changeable volume portion and an additional energy storage device.

Additional Electronic Sensor Load Measuring. As shown in FIG. 47, very thin flexible load sensor 4703 can be used to measure the cuff tension at each end of the implant along the length between the balloon and vessel, and between the cuff and the vessel. Multiple load sensors could be used to validate the cuff tension then allowing adjusting each end of cuff tension to balance the cuff load at each end of the device. The sensor can connect to their own electronic circuit wired to a data recording system, or could be connected to the implanted monitoring system for temporary use at implant, or if the sensors are embedded/sealed and fixed to the device, they could be used to measure device stability post implantation. Also shown in FIG. 47, is the clamping and suturing of the cuff ends 4704 and 4705, where one side is shown with the cuff ends adjusted to be fixed at matching adjoining ends, and the other has a slight difference indicating this end may be either too loose or fitted with the same load around a larger vessel diameter. The load sensors can therefore quantitate and confirm a balanced cuff tension and load at each end. Prior to attaching the cuff, the ascending aorta and pulmonary artery are isolated along the marked area 4701. The cuff 4702 is then slid through this slot as indicated by the arrow, sliding the cuff between the ascending aorta (AA) and pulmonary artery and back around the AA to the other cuff end. Once the cuff and device are adjusted, the cuff ends are sutured and any excess cuff length is trimmed off. The device is now ready for use as shown in 4706. The device can also be used on the pulmonary artery as indicated in 4707 by sliding the cuff through a similar vessel isolation slot 4701. Two devices can be used on both the ascending aorta 4706 and the pulmonary artery 4707 on the same patient. In this case, a cuff may be used that wraps around both vessels which are connected at the AA to PA isolated point. Two complete cuffs that overlap at the AA/PA contact area is also possible, or alternatively both devices could be attached to one cuff wrapping both the AA and PA without isolating the connective tissue joining the AA and PA.

The devices and methods described above can be used to address the following difficulties: hypertension and aortic stiffening by means of the above described compliant prothesis in stentgraft or graft form, and the tubular wall compliance device.

In respect of the above described embodiments, a chemical agent might be added to shrink or constrict bio-polymers in the devices described above prior to deployment. The above described technology can also be applied to other associated medical applications including but not limited to: coronary bypass grafting prostheses (in exclusion or inclusion of all grafting (vein, xeno, synthetic, biodegradable, tissue engineered substitutes); stenting applications; dialysis; others.

The embodiments described above serve to enhance the secondary heart pump action of the cardiovascular system. They have a time dependent pressure dampening effect during systole, and a time dependent pressure discharge during diastole, thus a counter pulsation enhancement, lower heart workload and enhancing blood flow during diastole, increasing aortic and coronary artery blood flow. Our device studies in humans have also shown improvements in cardiac output and reduced heart rates consistent with treating aortic stiffening.

The systems can be particularly useful for the treatment of hypertension, and various stages of heart failure from mild to severe, and where indicated for the treatment aortic aneurysms, and for the unloading of a vessel or luminal passage.

These embodiments improve the prior art by: increasing efficiency; being self-powered; being less complex, being more reliable, and highly cost effective, being less invasive to implant giving faster procedure time and quicker patient recovery and less cost by comparison to prior art systems and their use, and having features to reduce implantation complications, a secure, safe and stable device attached to a vessel, and features to control, monitor, log data, for improving and adjusting performance during long term implantation and use. It will be understood that the invention disclosed and defined herein extends to all alternative combinations of two or more of the individual features mentioned or evident from the text or drawings. All of these different combinations constitute various alternative aspects of the invention. The foregoing describes embodiments of the present invention and modifications, obvious to those skilled in the art can be made thereto, without departing from the scope of the present invention.

1-48. (canceled)

49. A treatment device for operating with a wall of a vessel in a human or animal body, comprising:
a changeable volume portion adapted to attach to the vessel and modify a volume of the vessel;
a mechanical or electronic-mechanical energy storage device adapted to function with the changeable volume portion such that, in use, the changeable volume portion decreases the vessel volume when applied, allowing the volume of the vessel to increase during systole and dampen pressure by the changeable volume portion and energy storage device absorbing energy, subsequently releasing the absorbed energy during diastole to cause the changeable volume portion to decrease the vessel volume; and
additional mechanical and electronic device and sensor components to control a load applied to the wall of the vessel and the treatment device to allow electronic dynamic dampening control and mechanical or electronic-mechanical energy harvesting and discharging means to achieve independent and dynamically controlled charge and discharge modes.

50. The device as claimed in claim 49, wherein the changeable volume portion is constructed at least in part from an elastomeric material, the elastomeric material being the energy storage means.

51. The device as claimed in claim 49, wherein the changeable volume portion is a graft or a stent graft, or a part thereof and the energy storage means is an elastomeric material or deformable stent member which forms the graft, the stent graft part or the part thereof.

52. The device as claimed in claim 49, wherein the changeable volume portion and the energy storage means are adjusted to a threshold or reference position, volume, or pressure, the device being adjustable via an attached port at time of implantation and during use.

53. The device as claimed in claim 49, wherein media with which the changeable volume portion is primed with one or more of the following media: a bio-compatible fluid; liquid silicone; liquid saline; water; a liquid containing a contrast agent which is x-ray viewable; a gel or other solution that expands with temperature to a final operating volume at 37° degrees Celsius; elastin; collagen; elastin and collagen in combination; air; carbon dioxide, helium; nitrogen; or a gas.

54. The device as claimed in claim 49, wherein media with which the energy storage means is primed is one or more of the following compressible media: air, carbon dioxide, helium, nitrogen, gas, other compressible media.

55. The device as claimed in claim 49, where the performance of the device can be monitored by electronic sensors mounted in an attached port and or mounted in the changeable volume portion and energy storage device.

56. The device as claimed in claim 49, wherein the electronic device and sensor components are electrically powered by an attached implanted battery, an attached electronic energy harvesting circuit, an attached implanted induction coil charged via inductive power delivered by an external coil, or via electrical power connected with an electrical subcutaneous port and electrical power needle, wherein the electronic device and sensor components are connected to an electronic communications circuit via analogue to digital conversion or via a digital connection, using an electronic communication circuit that can send data electronically via RF, blue tooth, or an electrical subcutaneous port to an external receiver to log and record data.

57. The device as claimed in claim 49, wherein the device has an attached tag or tape for deploying and positioning the device around the vessel, the device being flexible and compressible to fit into a deployment tool to fit into a standard endoscopy TROCAR port, allowing for a surgical instrument to access the device tag via an additional endoscopy port to unload the device, the endoscopy ports inserted in the intercostal spaces or tissues in proximity to the vessel.

58. The device as claimed in claim 49, that uses load sensors attached to an electronic circuit and a data logger to quantitate the cuff tension and balloon to vessel coupling, allowing adjustment of the cuff ends to balance the load at each side on the device at time of implanting and for monitoring device status and performance.

59. The device as claimed in claim 49, wherein the changeable volume portion is a cuff member comprising an inflatable portion, the cuff member and the inflatable portion being able to be positioned around or in the vessel.

60. The device as claimed in claim 49, wherein the device contains an adjustable attachment tensioner.

61. The device as claimed in claim 49, wherein the device contains an adjustable outer cushion to protect surrounding vessels and tissues.

62. The device as claimed in claim 52, wherein the port is attached to syringe piston where the piston is incrementally stepped with a stepper motor to increase or decrease the device position, volume, or pressure threshold or reference to an adjusted operational level.

63. The device as claimed in claim 49, where the connected tubing is wire reinforced and comprises multiple lumens allowing for one or more mechanical and electrical connections, comprising insulated electrical conductors to power and receive data from attached sensors, and for independently adjusting the position, volume, media or pressure of: the changeable volume portion; an attachment tensioner; an outer protection cushion; position of attached syringe piston.

64. A cuff, comprising:
a changeable volume portion configured to operate with a wall of a vessel in a human or animal body, the cuff is adapted for attachment to the vessel and for modifying the volume of the vessel;
a mechanical or electronic-mechanical energy storage device adapted to function with the changeable volume portion such that, in use, the changeable volume portion decreases the vessel volume when applied, allowing the volume of the vessel to increase during systole and dampen pressure by the changeable volume portion and energy storage device absorbing energy, subsequently releasing the absorbed energy during diastole to cause the changeable volume portion to decrease the vessel volume; and
additional mechanical and electronic device and sensor components to control a load applied to the wall and device to allow electronic dynamic dampening control and mechanical or electronic-mechanical energy harvesting and discharging means to achieve independent and dynamically controlled charge and discharge modes.

65. The cuff as claimed in claim 64, wherein the cuff being of an elongated and thin form having a first portion which is convergent then divergent in a longitudinal direction of the cuff, the cuff comprising a second portion adjacent, near to, or in the vicinity of, the first portion, the second portion having at least one aperture.

66. The cuff as claimed in claim 64, wherein the cuff has at least one aperture cut out for shaping the cuff around the inner radius of a curved vessel, the aperture being convergent and divergent in at least one section of the cuff.

67. The cuff as claimed in claim 64, that uses at least two end flap cuff configurations for independent tensioning of cuff to vessel.

68. The cuff as claimed in claim 64, that uses a cuff with a cut out window to improve the range of the changeable volume.

69. The cuff as claimed in claim 64, where the cuff window contains an attached deformable sheet.

70. The cuff as claimed in claim 64, wherein the cuff is attached using a double bar cuff attachment, a single bar cuff attachment, or a split cuff bar attachment.

71. The cuff as claimed in claim 64, wherein the cuff is attached by using side flaps connected to the sides of the changeable volume portion for independent tensioning of the cuff to the changeable volume portion.

72. A method for treating a vessel, comprising:
preparing a patient;
identifying a site in the vessel requiring treatment;
positioning an implantable treatment device against a portion of tubular or sac wall of the vessel at the site for operating with a wall of a vessel in a human or animal body, the implantable treatment device comprising:
a changeable volume portion adapted to attach to the vessel and modify the volume of the vessel;
a mechanical or electronic-mechanical energy storage device which is adapted to function with the changeable volume portion such that, in use, the changeable volume portion decreases the vessel volume when applied, allowing the volume of the vessel to increase during systole and dampen pressure by the changeable volume portion and energy storage device absorbing energy, subsequently releasing the absorbed energy during diastole to cause the changeable volume portion to decrease the vessel volume; and
additional mechanical and electronic device and sensor components to control the load applied to the wall and device to allow electronic dynamic dampening control and mechanical or electronic-mechanical energy harvesting and discharging means to achieve independent and dynamically controlled charge and discharge modes.

73. The method as claimed in claim 72, further comprising applying the treatment device to an ascending aorta by isolating it from a pulmonary artery.

74. The method as claimed in claim 72, where the treatment device is applied to both the ascending aorta and the pulmonary artery.

75. The method as claimed in claim 72, wherein the treatment device is applied to multiple vessels comprising the ascending and descending vessels attached to both the right and left sides of the heart.

76. The method as claimed in claim 72, wherein:
the electronic device and sensor components are connected to an electronic communications circuit via analogue to digital conversion or via a digital connection using an electronic communication circuit that can send data electronically via a wireless communication medium, or an electrical subcutaneous port to an external receiver to log and record data;
the electronic device and sensor components are electrically powered by an attached implanted battery, an attached electronic energy harvesting circuit, an attached implanted induction coil charged via inductive power delivered by an external coil, or via electrical power connected with an electrical subcutaneous port and electrical power needle;
the connected tubing is wire reinforced and comprises multiple lumens allowing for one or more mechanical and electrical connections, comprising insulated electrical conductors to power and receive data from attached sensors, and for independently adjusting the position, volume, media or pressure of: the changeable volume portion; an attachment tensioner; an outer protection cushion; and the position of attached syringe piston.
